(19) United States
(12) Patent Application Publication
(10) Pub. No.: US 2015/0305999 A1
(43) Pub. Date: Oct. 29, 2015

(54) COMPOSITION COMPRISING A DICARBONYL DERIVATIVE AND METHOD FOR STRAIGHTENING THE HAIR USING THIS COMPOSITION

(71) Applicant: L'OREAL, Paris (FR)

(72) Inventor: Nicolas DAUBRESSE, La Celle Saint Cloud (FR)

(21) Appl. No.: 14/441,343

(22) PCT Filed: Nov. 8, 2013
(86) PCT No.: PCT/FR2013/052691

§ 371 Date: May 7, 2015

(30) Foreign Application Priority Data
Nov. 9, 2012 (FR) 1260681

(51) Int. Cl. A61K 8/43; A45D 2/00; A61Q 5/06; A45D 7/06; A61K 8/365; A61K 8/42

(52) U.S. Cl. CPC A61K 8/43; A61K 8/365; A61K 8/42; A61Q 5/06; A45D 7/06; A45D 2/001; A61K 2800/805; A61K 2800/591

(57) ABSTRACT

The present invention relates to a cosmetic composition comprising:
one or more dicarbonyl compounds corresponding to formula (I) below, and/or derivatives thereof and/or hydrates thereof and/or salts thereof:

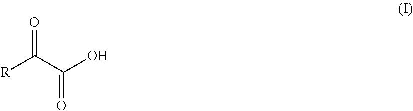

one or more amine-generating derivatives chosen from the compounds of formula (II) below, salts thereof, tautomers thereof or hydrates thereof:

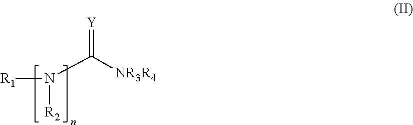

The invention also relates to a process for straightening keratin fibers, especially the hair, which comprises the application to the hair of the composition of the invention, followed by a straightening step using a straightening iron at a temperature of at least 150° C., preferably ranging from 150 to 250° C.

The present invention relates to a cosmetic composition, especially a hair composition, based on one or more particular dicarbonyl derivatives, and also to a process for straightening keratin fibers, especially the hair, using this composition.

In the hair field, consumers wish to have available compositions which make it possible to introduce a temporary change to their head of hair, while targeting good persistence of the effect produced. In general, it is desired for the change to withstand shampooing for at least fifteen days or even more, depending on the nature of said change.

Treatments already exist for modifying the color or shape of the hair and also, to a certain extent, the texture of the hair. One of the treatments known for modifying the texture of the hair consists in combining heat and a composition comprising formaldehyde. This treatment is especially effective for imparting a better appearance to damaged hair and/or for treating long hair and curly hair.

The action of formaldehyde is associated with its ability to crosslink proteins by reaction on the nucleophilic sites thereof. The heat used may be that of an iron (flat tongs or crimping iron), the temperature of which may generally be up to 200° C. or more. However, it is increasingly sought to avoid the use of such substances, which may prove to be aggressive to the hair and other keratin materials.

Patent application WO 2011/104 282 thus proposed a novel process for semi-permanently straightening the hair, which consists in applying an α-keto acid solution to the hair for 15 to 120 minutes, then drying and, finally, straightening the head of hair with an iron at a temperature of about 200° C. The α-keto acid employed is preferably glyoxylic acid.

However, it has been noted that the use of glyoxylic acid can result in some significant limitations; in particular, at high concentration it may not be well tolerated, in particular when the scalp is sensitive and/or irritated. Its volatility, amplified by the use of heat (iron), can also be a problem. Moreover, cosmetic formulations at acid pH may degrade the hair and/or modify the color thereof.

It is already known practice to use glyoxylic acid esters in hair compositions, in particular in hair dye compositions, as described in document DE19859722, and in reducing compositions, as described in document DE19860239.

However, the efficacy of these compositions is not yet sufficient.

The aim of the invention is to develop a straightening/relaxing composition which is stable over time and which makes it possible to straighten/relax and/or reduce the volume of the hair in an efficient and persistent manner while limiting damage to the hair, while at the same time retaining comfort at the time of application for the user of the composition, but also for the hairdresser who applies it.

Thus, one subject of the present invention is a cosmetic composition comprising:
one or more dicarbonyl compounds corresponding to formula (I) below, and/or derivatives thereof and/or hydrates thereof and/or salts thereof:

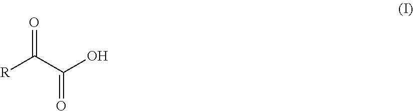

in which formula (I):
R represents an atom or group chosen from i) hydrogen, ii) carboxy —C(O)—OH, iii) linear or branched C₁-C₆ alkyl which is optionally substituted, preferably with at least one hydroxyl —OH radical, carboxy —C(O)—OH radical or halogen such as Br; iv) optionally substituted phenyl, v) optionally substituted benzyl, iv) and v) preferably being optionally substituted with at least one —OH or —C(O)—OH radical; vi) an indolyl radical and vii) an imidazolylmethyl radical and tautomers thereof such as

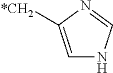

with * representing the part linked to the rest of the molecule,
one or more amine-generating derivatives chosen from the compounds of formula (II) below, salts thereof, tautomers thereof or hydrates thereof:

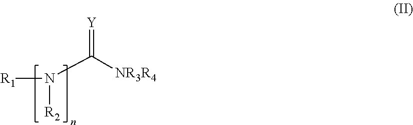

in which formula (II):
Y represents an atom or group chosen from i) oxygen, ii) sulfur, iii)N—R′; preferably, Y represents an oxygen atom or N—R′, more preferentially Y═O;
R′ representing an atom or group chosen from i) hydrogen, ii) a linear or branched, cyclic or acyclic C₁-C₅ alkyl or alkenyl radical, this radical being optionally substituted with one or more radicals chosen from the following radicals: hydroxyl, (di)(C₁-C₆)(alkyl)amino such as dimethylamino, carboxyl, halogen, C₆-C₁₈ aryl, carboxamide and N-methylcarboxamide; preferably, R′ represents a hydrogen atom or a group (C₁-C₆)alkyl optionally substituted with one or more (di)(C₁-C₆)(alkyl)amino and/or carboxyl radicals;
n=0 or 1, preferably 1,
R₁, R₂, R₃ and R₄, which may be identical or different, represent i) a hydrogen atom, a linear or branched, saturated or unsaturated, optionally substituted C₁-C₃₀ hydrocarbon-based chain, optionally interrupted and/or terminated at one or both of its ends with one or more divalent groups or combinations thereof chosen from:
   —N(R)—, —N⁺(R)(R′)—, —O—, —S—, —C(O)—, —S(O)—, —S(O)₂— with R, R′, which may be identical or different, are chosen from hydrogen, C₁-C₄ alkyl, (C₁-C₄)hydroxyalkyl and (di)(C₁-C₄)(alkyl)aminoalkyl radicals; preferentially with one or more divalent groups or combinations thereof chosen from: —N(R)—, —O— and —C(O)—, —N(R)—, —N⁺(R)(R′)—, —O—, —S—, —C(O)—, —S(O)—, —S(O)₂—
   an optionally substituted (hetero)cycloalkyl or optionally substituted (hetero)aryl radical;
      or else
R₁ and R₂ and/or R₃ and R₄ form, together with the nitrogen atom that bears them, an optionally substituted heterocycloalkyl group or an optionally substituted heteroaryl group; or else
R₁ and R₃ form, together with the nitrogen atom that bears them, an optionally substituted heterocycloalkyl group.

Unless otherwise mentioned:

The “(hetero)aryl” radicals, or the “(hetero)aryl” part of a radical when they are optionally substituted, said radicals may then be substituted on a carbon atom, with an atom or group chosen from: i) C₁-C₁₆, preferably C₁-C₈ alkyl, optionally substituted with one or more radicals chosen from the following radicals: hydroxyl, C₁-C₂ alkoxy, (poly)(C₂-C₄)hydroxyalkoxy, acylamino, amino substituted with one or two identical or different C₁-C₄ alkyl radicals, optionally bearing at least one hydroxyl group, or the two radicals possibly forming, with the nitrogen atom to which they are attached, a 5- to 7-membered and preferably 5- or 6-membered, saturated or unsaturated, optionally substituted heterocycle optionally comprising another nitrogen or non-nitrogen heteroatom; ii) halogen; iii) hydroxyl; iv) C₁-C₂ alkoxy; v) (poly)(C₂-C₄)hydroxyalkoxy; vi) amino; vii) 5- or 6-membered heterocycloalkyl; viii) 5- or 6-membered, optionally cationic heteroaryl, preferentially imidazolium, and optionally substituted with a (C₁-C₄) alkyl radical, preferentially methyl; ix) amino substituted with one or two identical or different C₁-C₆ alkyl radicals optionally bearing at least one hydroxyl group or amino group optionally substituted with one or two optionally substituted C₁-C₃ alkyl radicals; x) acylamino (—N(R)—C(O)—R′) in which the radical R is a hydrogen atom, a C₁-C₄ alkyl radical optionally bearing at least one hydroxyl group and the radical R′ is a C₁-C₂ alkyl radical; xi) carbamoyl ((R)₂N—C(O)—) in which the radicals R, which may be identical or different, represent a hydrogen atom, a C₁-C₄ alkyl radical optionally bearing at least one hydroxyl group; xii) alkylsulfonylamino (R′—S(O)₂—N(R)—) in which the radical R represents a hydrogen atom, a C₁-C₄ alkyl radical optionally bearing at least one hydroxyl group and the radical R′ represents a C₁-C₄ alkyl radical or a phenyl radical; xiii) aminosulfonyl ((R)₂N—S(O)₂—) in which the radicals R, which may be identical or different, represent a hydrogen atom, a C₁-C₄ alkyl radical optionally bearing at least one hydroxyl group, xiv) carboxyl in acid or salified form (preferably with an alkali metal or a substituted or unsubstituted ammonium); xv) cyano; xvi) nitro or nitroso; xvii) polyhaloalkyl, preferentially trifluoromethyl;

The “(hetero)cyclic” or “(hetero)cycloalkyl” radicals when they are optionally substituted may be substituted with at least one atom or group chosen from: i) hydroxyl; ii) C₁-C₄ alkoxy, (poly)(C₂-C₄)hydroxyalkoxy; iii) C₁-C₄ alkyl; iv) alkylcarbonylamino (R—C(O)—N(R′)—) in which the radical R′ is a hydrogen atom, a C₁-C₄ alkyl radical optionally bearing at least one hydroxyl group and the radical R is a C₁-C₂ alkyl radical or amino radical optionally substituted with one or two identical or different C₁-C₄ alkyl groups which themselves optionally bear at least one hydroxyl group, said alkyl radicals possibly forming, with the nitrogen atom to which they are attached, a saturated or unsaturated, optionally substituted 5- to 7-membered heterocycle optionally comprising at least one other nitrogen or non-nitrogen heteroatom; v) alkylcarbonyloxy (R—C(O)—O—) in which the radical R is a C₁-C₄ alkyl radical, amino group optionally substituted with one or two identical or different C₁-C₄ alkyl groups which themselves optionally bear at least one hydroxyl group, said alkyl radicals possibly forming, with the nitrogen atom to which they are attached, a saturated or unsaturated, optionally substituted 5- to 7-membered heterocycle optionally comprising at least one other nitrogen or non-nitrogen heteroatom; vi) carboxyl in salified or non-salified form, or alkoxycarbonyl (R-G-C(O)—) in which the radical R is a hydrogen atom or a C₁-C₄ alkoxy radical, G is an oxygen atom or an amino group optionally substituted with a C₁-C₄ alkyl group which itself optionally bears at least one hydroxyl group, said alkyl radical possibly forming, with the nitrogen atom to which it is attached, a saturated or unsaturated, optionally substituted 5- to 7-membered heterocycle optionally comprising at least one other nitrogen or non-nitrogen heteroatom; vii) halogen;

The “(hetero)cyclic” or “(hetero)cycloalkyl” radicals, or a non-aromatic part of a (hetero)aryl radical, when they are optionally substituted, may also be substituted with one or more oxo groups;

A hydrocarbon-based chain is “unsaturated” when it comprises one or more double bonds and/or one or more triple bonds, which may be conjugated or non-conjugated;

An “aryl” radical represents a fused or non-fused monocyclic or polycyclic carbon-based group comprising from 6 to 22 carbon atoms, and in which at least one ring is aromatic; preferentially, the aryl radical is a phenyl, biphenyl, naphthyl, indenyl, anthracenyl or tetrahydronaphthyl;

A “heteroaryl radical” represents an optionally cationic, 5- to 22-membered, monocyclic or fused or non-fused polycyclic group, comprising from 1 to 6 heteroatoms chosen from nitrogen, oxygen, sulfur and selenium, at least one ring of which is aromatic; preferentially, a heteroaryl radical is chosen from acridinyl, benzimidazolyl, benzobistriazolyl, benzopyrazolyl, benzopyridazinyl, benzoquinolyl, benzothiazolyl, benzotriazolyl, benzoxazolyl, pyridinyl, tetrazolyl, dihydrothiazolyl, imidazopyridyl, imidazolyl, indolyl, isoquinolyl, naphthoimidazolyl, naphthoxazolyl, naphthopyrazolyl, oxadiazolyl, oxazolyl, oxazolopyridyl, phenazinyl, phenoxazolyl, pyrazinyl, pyrazolyl, pyrilyl, pyrazoyltriazyl, pyridyl, pyridinoimidazolyl, pyrrolyl, quinolyl, tetrazolyl, thiadiazolyl, thiazolyl, thiazolopyridinyl, thiazoylimidazolyl, thiopyrylyl, triazolyl, xanthyl and the ammonium salt thereof;

A “heterocyclic” or “heterocycloalkyl” radical is a fused or non-fused, 5- to 22-membered monocyclic or polycyclic radical which may contain one or more unsaturations but is non-aromatic, comprising from 1 to 6 heteroatoms chosen from nitrogen, oxygen, sulfur and selenium, such as morpholino, piperidino, piperazino, tetrahydrofuryl or pyrrolidyl;

A “cycloalkyl” radical is a monocyclic or polycyclic, fused or non-fused, 5- to 22-membered hydrocarbon-based radical which may contain one or two unsaturations but is non-aromatic, such as cyclohexyl, cyclopentyl or cyclobutyl;

An “alkyl” radical is a linear or branched C₁-C₂₀, preferably C₁-C₈ hydrocarbon-based radical such as methyl or ethyl;

The term “optionally substituted” applied to the “alkyl”, “alkylene” or “alkenylene” radical or to “a hydrocarbon-based chain” implies that said radical may be substituted with one or more radicals chosen from halogen atoms and the following radicals: i) hydroxyl, ii) C₁-C₄ alkoxy, iii) acylamino, iv) amino optionally substituted with one or two identical or different C₁-C₄ alkyl radicals, said alkyl radicals possibly forming, with the nitrogen atom that bears them, a 5- to 7-membered heterocycle optionally comprising another nitrogen or non-nitrogen heteroatom, v) or a quaternary ammonium group —N⁺R′R″R′″, M⁻ for which R′, R″ and R′″, which may be identical or different, represent a hydrogen atom or a C₁-C₄ alkyl group, or alternatively —N⁺R′R″R′″ forms a heteroaryl such as imidazolium optionally substituted with a C₁-C₄ alkyl group, and M⁻ represents the counterion of the organic or mineral acid or of the corresponding halide, vi) oxo.

An “alkoxy radical” is an alkyl-oxy radical for which the alkyl radical is a linear or branched C₁-C₁₆ and preferentially C₁-C₈ hydrocarbon-based radical.

When the alkoxy group is optionally substituted, this implies that the alkyl group is optionally substituted as defined hereinabove.

A subject of the invention is also a process for straightening keratin fibers, especially the hair, which comprises the application to the hair of the composition of the invention, followed by a straightening step using a straightening iron at a temperature of at least 150° C., preferably ranging from 150 to 250° C.

The composition of the invention is stable. The composition of the invention, and the process for treating keratin fibers using it allow good straightening of keratin fibers while limiting damage to these keratin fibers, even when the application of the composition is followed by a heat treatment, in particular using a hair-straightening iron, and have an appreciated wear quality, in particular without excessive vaporization of the composition at the time of straightening. The composition and the process for treating keratin fibers as claimed in the invention also make it possible to limit the change in the color of the fibers and also the problems of breaking of the fibers such as the hair. The composition and the process of the invention will also improve the physical properties of the hair, by durably reducing the frizziness effect of the hair.

In that which follows, the expression “at least one” is equivalent to the expression “one or more”.

Preferably, the composition according to the invention does not comprise any coloring agent or any reducing agent.

According to the present invention, the term “coloring agents” is intended to mean agents for coloring keratin fibers, such as direct dyes, pigments or oxidation dye precursors (bases and couplers). If they are present, their content does not exceed 0.001% by weight relative to the total weight of the composition. Specifically, at such a content, only the composition would be dyed, i.e. no dyeing effect would be observed on the keratin fibers.

It is recalled that oxidation dye precursors, oxidation bases and couplers are colorless or sparingly colored compounds, which, via a condensation reaction in the presence of an oxidizing agent, give a colored species. With regard to direct dyes, these compounds are colored and have a certain affinity for keratin fibers.

According to the present invention, the term “reducing agent” is intended to mean an agent that is capable of reducing the disulfide bonds of the hair, such as compounds chosen from thiols, alkaline sulfites, hydrides and phosphines.

In the present invention, the dicarbonyl compounds of formula (I) or derivatives thereof may be in free form, but also in their hydrate forms or in the form of their salts, preferably in free form or in the form of hydrates. As “derivatives” of the dicarbonyl compounds of formula (I), mention may be made of esters of the carboxyl group(s), amides of the carboxyl group(s), and (thio)acetals and hemi(thio)acetals of the carbonyl function(s) of the compounds of formula (I), in free form or optionally in the form of salts or of hydrates, preferably in free form or in the form of hydrates.

The esters and amides may be synthesized via conventional esterification or amidation processes, from the corresponding acids well known to those skilled in the art.

The esters are obtained, for example, from the acids of formula (I) and from a mono- or polyalcohol.

The term “mono- or polyalcohol” is intended to mean an organic compound comprising one hydroxyl group (monoalcohol) or at least two hydroxyl groups (polyalcohol or polyol); said hydroxylated organic compound possibly being aliphatic, acyclic, linear or branched, or (hetero)cyclic, such as sugars (monosaccharides or polysaccharides) or sugar alcohols.

More particularly, the polyalcohol comprises from 2 to 100 hydroxyl groups, preferentially from 2 to 20 hydroxyl groups, even more preferentially from 2 to 10 hydroxyl groups and better still 2 or 3 hydroxyl groups.

Preferably, the mono- or polyalcohol is chosen from methanol, ethanol, propanol, isopropanol, butanol, hexanol, ethylene glycol, glycerol, dihydroxyacetone, glucose, sorbitol and menthol.

The amides are obtained, for example, from the acids of formula (I) and from an organic mono- or polyamine.

The term “mono or polyamine” is intended to mean an organic compound comprising one amino group (monoamine) or at least two (and preferably from 2 to 100, better still from 2 to 20) amine groups; said organic compound possibly being aliphatic, acyclic, linear or branched or (hetero)cyclic.

The term “amino” group is intended to mean a primary amine group —NH₂, or a secondary amine group >NH.

The mono or polyamine is preferably aliphatic.

This amine is preferably chosen from methylamine, ethylamine, propylamine, isopropylamine, butylamine, hexylamine, monoethanolamine, monopropanolamine, propane-1,2,3-triamine and diaminoacetone.

The (thio)acetals and hemi(thio)acetals of the acids of formula (I) may be obtained, for example, from the reaction of alcohols, for the acetals or hemiacetals, or of thiols, for the thioacetals or hemithioacetals, with blocked forms of the acids, followed by hydrolysis. The alcohols may be the same as those mentioned for the esters. The thiols may be equivalents (referred to as mono- or polythiols) to the mono- or polyalcohols mentioned above, except for the fact that the hydroxyl function(s) of said mono- or polyalcohols are replaced with one or more thiol functions SH of the mono- or polythiols. The acetals or thioacetals may also be cyclic (thio)acetals.

Mention may in particular be made of dimethoxyacetic acid, diethoxyacetic acid, 1,3-dioxane-2-carboxylic acid and 1,3-dioxolane-2-carboxylic acid.

The salts may be salts derived from the interaction of the compounds of formula (I) with acids or bases, it being possible for the acids or bases to be of organic or mineral nature.

Preferably, the salts are salts derived from the interaction of the compounds of formula (I) with bases. Alkali metal or alkaline-earth metal salts, and in particular the sodium salts, will in particular be mentioned.

Preferably, the dicarbonyl derivative(s) corresponding to formula (I) and/or derivatives thereof and/or hydrates thereof and/or salts thereof are chosen from the dicarbonyl derivatives corresponding to formula (I) in which R represents i) a hydrogen atom or ii) a linear or branched C₁-C₆ alkyl group optionally substituted with a carboxy group.

More preferably, they are chosen from glyoxylic acid and pyruvic acid, a derivative thereof and hydrates thereof or salts thereof and more preferentially from glyoxylic acid, derivatives thereof and the hydrate forms of these compounds.

Preferentially, the dicarbonyl compound(s) of formula (I) of the invention are chosen from glyoxylic acid and derivatives thereof and the hydrate forms of these compounds.

Mention may be made of glyoxylic acid and also the hydrate form thereof (HO)₂CH—C(O)—OH, for instance the glyoxylic acid as a 50% aqueous solution sold by the company Merck.

Glyoxylic acid derivatives that may be mentioned include glyoxylic acid esters, glyoxylic acid amides, glyoxylic acid (thio)acetals and hemi(thio)acetals, and glyoxylic acid ester (thio)acetals and hemi(thio)acetals.

The glyoxylic acid esters are obtained, for example, from glyoxylic acid and a mono- or polyalcohol, especially those mentioned previously.

Preferably, the mono- or polyalcohol is chosen from methanol, ethanol, propanol, isopropanol, butanol, hexanol, ethylene glycol, glycerol, dihydroxyacetone, glucose, sorbitol and menthol.

Esters that may in particular be mentioned include methyl glyoxylate, ethyl glyoxylate, glyceryl glyoxylate, dihydroxyacetone glyoxylate, glyceryl diglyoxylate or triglyoxylate, sorbitol mono-, di- or triglyoxylate, glucose mono-, di- or triglyoxylate, menthyl glyoxylate, and the acetals, hemiacetals and hydrates thereof.

The glyoxylic acid amides are, for example, obtained from glyoxylic acid and an organic mono or polyamine.

This amine is preferably chosen from methylamine, ethylamine, propylamine, isopropylamine, butylamine, hexylamine, monoethanolamine, monopropanolamine, propane-1,2,3-triamine and diaminoacetone.

Mention may in particular be made of glyoxylic acid N-β-hydroxyethylamide and glyoxylic acid N-γ-hydroxypropylamide and the (thio)acetals, hemi(thio)acetals and hydrates thereof.

The glyoxylic acid (thio)acetals and hemi(thio)acetals may be obtained, for example, from the reaction of alcohols of thiols, for the thioacetals or hemithioacetals, with blocked forms of glyoxylic acid, followed by hydrolysis. The alcohols may be the same as those mentioned for the esters. The thiols may be equivalents (referred to as mono- or polythiols) to the mono- or polyalcohols mentioned above, except for the fact that the hydroxyl function(s) of said mono- or polyalcohols are replaced with one or more thiol functions SH of the mono- or polythiols. The acetals or thioacetals may also be cyclic (thio)acetals.

According to a particularly preferred embodiment, the compound of formula (I) is glyoxylic acid in hydrate form.

According to one embodiment, the composition of the invention comprises from 0.5% to 15% of one or more dicarbonyl derivatives corresponding to formula (I) and/or of a derivative thereof and/or of hydrate forms thereof and/or salts thereof, preferably from 3% to 15% and preferentially from 5% to 10% by weight relative to the total weight of the composition.

According to the invention, the composition comprises one or more amine-generating derivatives corresponding to formula (II) described previously.

According to a particular embodiment of the invention, the compounds of formula (II) are such that R₁, R₂, R₃ and R₄ represent, independently of each other:

(i) a hydrogen atom, (ii) a linear or branched, cyclic or acyclic C₁-C₅ alkyl or alkenyl radical, (iii) a C₁-C₅ alkoxy radical, (iv) a 5- to 18-membered (hetero)aryl radical, preferably C₆-C₁₈ aryl, (v) a 5- to 8-membered (hetero)cyclic radical; these radicals (ii) to (v) being optionally substituted in particular with one or more radicals chosen from the following radicals: hydroxyl, (C₁-C₄)alkyl, (di)(C₁-C₆)(alkyl)amino such as dimethylamino, carboxyl, halogen, C₆-C₁₈ aryl, carboxamide and N-methylcarboxamide;
it being understood that:
when R₁, R₂ and R₃ represent a hydrogen atom, R₄ may denote a radical from among: carboxamide, methoxy, ethoxy, 1,2,4-triazolyl, cyclopentyl, (C₁-C₆)alkylcarbonyl such as acetyl, (C₁-C₆)alkoxycarbonyl such as methoxycarbonyl or ethoxycarbonyl, —C(O)—C(H)═C(H)—C(O)—OH, phenyl optionally substituted with a chlorine atom or a hydroxyl, benzyl or 2,5-dioxo-4-imidazolidinyl radical;
when R₁ and R₃ represent a hydrogen atom, R₂ may represent a hydrogen atom or a methyl or ethyl radical and R₄ may represent an acetyl radical;
when R₁═R₂═H, R₃ and R₄ can form, with the nitrogen atom that bears them, a piperidine, 3-methylpyrazole, 3,5-dimethylpyrazole or maleimide ring;
R₁ and R₂ and also R₃ and R₄ can form, with the nitrogen atom that bears them, an imidazole ring;
R₁ and R₃ or R₂ and R₄ together possibly constituting a radical chosen from the following divalent radicals: —CH₂—CH₂—, —CH═CH—, —CH₂—C(O)—, —C(O)—N(H)—, —CH═N—, —C(O)—C(O)—, —CH(OH)—CH(OH)—, —[HO(O)C]CH—CH—, —CH(OH)—C(O)—, —CH₂—CH₂—CH₂—, —CH₂—N(H)—C(O)—, —CH═C(CH₃)—C(O)—, —N(H)—C(O)—N(H)—, —CH₂—CH₂—C(O)—, —CH₂—N(CH₃)—CH₂—, —N(H)—CH₂—N(H)—, —C(O)—CH(CH₃)—CH₂—, —C(O)—CH₂—C(O)—, —C(O)—N(H)—C(O)—, —C(O)—CH[C(O)OH)—CH₂—, —C(O)—CH═C[C(O)OH)]—, —C(O)—CH═C(CH₃)—, —C(O)—C(NH₂)═CH—, —C(O)—C(CH₃)═N—, —C(O)—CH═CH—, —C(O)—CH═N— and —C(O)—N═CH—;
Y is as defined previously, preferably Y═O.

Preferably, R₁, R₂, R₃ and R₄, represent, independently of each other, an atom or group chosen from i) hydrogen, ii) C₁-C₃ alkyl, which is optionally substituted, preferably with a hydroxyl radical.

According to one variant, R₁ and R₃ or R₂ and R₄ together constitute a saturated or unsaturated C₁-C₃ alkylene chain which may be optionally substituted with a hydroxyl group.

They then form a ring with the group —N—C(Y)—N—.

Preferably, the amine-generating derivatives corresponding to formula (II) are chosen from the amine-generating derivatives corresponding to formula (II) in which Y represents an atom or group chosen from oxygen and NR.

According to one embodiment, Y represents an —NH group.

Preferably, when R′ represents a hydrogen atom, R₁ represents a hydrogen atom or a group —(CH₂)₃—CH(NH₂)—C(O)—OH.

When Y represents N—R′, the amine-generating derivative is preferably chosen from guanidine, guanidine carbonate, arginine and arginine hydrochloride.

According to another particular embodiment, Y represents an oxygen atom.

When Y represents an oxygen atom, the amine-generating derivative is preferably chosen from urea and urea derivatives.

The term “urea derivative” means any compound other than urea CO(NH₂)₂ itself, comprising in its chemical formula a carbonyl group simply bonded to two nitrogen atoms, i.e. a unit

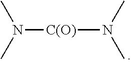

Preferably, urea or the urea derivative(s) are chosen from the compounds of formula (a) or (b), salts thereof, tautomers thereof or hydrates thereof:

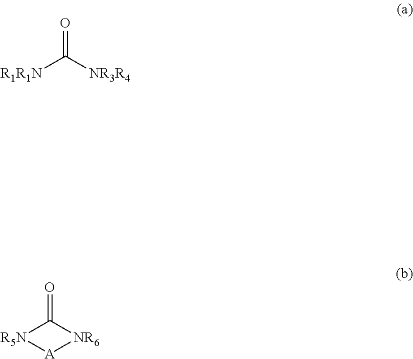

in which formulae (a) and (b):
R₁, R₂, R₃ and R₄ represent, independently of each other:
   (i) a hydrogen atom or
   (ii) a linear or branched, cyclic or acyclic C₁-C₅ alkyl or alkenyl radical, a C₁-C₅ alkoxy radical, (iii) a C₆-C₁₈ aryl radical, (iv) a 5- to 8-membered heterocyclic radical; these radicals being optionally substituted with one or more radicals chosen from the following radicals: hydroxyl, (C₁-C₄)alkyl, (di)(C₁-C₄)(alkyl)amino such as dimethylamino, carboxyl, halogen, C₆-C₁₈ aryl, carboxamide and N-methylcarboxamide;
   it being understood that:
when R₁, R₂ and R₃ represent a hydrogen atom, R₄ may denote a radical from among: carboxamide, methoxy, ethoxy, 1,2,4-triazolyl, cyclopentyl, (C₁-C₆)alkylcarbonyl such as acetyl, (C₁-C₆)alkoxycarbonyl such as methoxycarbonyl or ethoxycarbonyl, —C(O)—C(H)═C(H)—C(O)—OH, phenyl optionally substituted with a chlorine atom or a hydroxyl, benzyl or 2,5-dioxo-4-imidazolidinyl radical;
when R₁ and R₃ represent a hydrogen atom, R₂ may represent a hydrogen atom or a methyl or ethyl radical and R₄ may represent an acetyl radical;
when R₁═R₂═H, R₃ and R₄ can form, with the nitrogen atom that bears them, a piperidine, 3-methylpyrazole, 3,5-dimethylpyrazole or maleimide ring;
R₁ and R₂ and also R₃ and R₄ can form, with the nitrogen atom that bears them, an imidazole ring;
R₅ and R₆ represent, independently of each other,
   (iii) a hydrogen atom or
   (iv) a linear or branched, cyclic or acyclic C₁-C₅ lower alkyl, acyl or alkenyl radical, a C₁-C₅ alkoxy radical, a C₆-C₁₈ aryl radical, a 5- to 8-membered heterocyclic radical; these radicals being optionally substituted with a radical chosen from the following radicals: hydroxyl, amino, dimethylamino, carboxyl, halogen, C₆-C₁₈ aryl, carboxamide and N-methylcarboxamide;
A is a divalent radical chosen from the following divalent radicals: —CH₂—CH₂—, —CH═CH—, —CH₂—C(O)—, —C(O)—N(H)—, —CH═N—, —C(O)—C(O)—, —CH(OH)—CH(OH)—, —[HO(O)C]CH—CH—, —CH(OH)—C(O)—, —CH₂—CH₂—CH₂—, —CH₂—N(H)—C(O)—, —CH═C(CH₃)—C(O)—, —N(H)—C(O)—N(H)—, —CH₂—CH₂—C(O)—, —CH₂—N(CH₃)—CH₂—, —N(H)—CH₂—N(H)—, —C(O)—CH(CH₃)—CH₂—, —C(O)—CH₂—C(O)—, —C(O)—N(H)—C(O)—, —C(O)—CH[C(O)OH)—CH₂—, —C(O)—CH═C[C(O)OH)]—, —C(O)—CH═C(CH₃)—, —C(O)—C(NH₂)═CH—, —C(O)—C(CH₃)═N—, —C(O)—CH═CH—, —C(O)—CH═N— and —C(O)—N═CH—.

Among the compounds of formula (a) that are particularly preferred according to the invention, mention may be made of:
urea
methylurea
ethylurea
propylurea
n-butylurea
sec-butylurea
isobutylurea
tert-butylurea
cyclopentylurea
ethoxyurea
hydroxyethylurea
N-(2-hydroxypropyl)urea
N-(3-hydroxypropyl)urea
N-(2-dimethylaminopropyl)urea
N-(3-dimethylaminopropyl)urea
1-(3-hydroxyphenyl)urea
benzylurea
N-carbamoylmaleamide
N-carbamoylmaleamic acid
piperidinocarboxamide
1,2,4-triazol-4-ylurea
hydantoic acid
hydantoin
methyl allophanate
ethyl allophanate
acetylurea
hydroxyethyleneurea
2-(hydroxyethyl)ethyleneurea
diallylurea
chloroethylurea
N,N-dimethylurea
N,N-diethylurea
N,N-dipropylurea
cyclopentyl-1-methylurea
1,3-dimethylurea
1,3-diethylurea
1,3-bis(2-hydroxyethyl)urea
1,3-bis(2-hydroxypropyl)urea
1,3-bis(3-hydroxypropyl)urea
1,3-dipropylurea
ethyl-3-propylurea
sec-butyl-3-methylurea
isobutyl-3-methylurea
cyclopentyl-3-methylurea
N-acetyl-N′-methylurea
trimethylurea
butyl-3,3-dimethylurea
tetramethylurea and
benzylurea.

Among the compounds of formula (b) that are particularly preferred according to the invention, mention may be made of:
parabanic acid
1,2-dihydro-3-H-1,2,4-triazol-2-one
barbituric acid
uracil
1-methyluracil
3-methyluracil
5-methyluracil
1,3-dimethyluracil
5-azauracil
6-azauracil
5-fluorouracil
6-fluorouracil
1,3-dimethyl-5-fluorouracil
5-aminouracil
6-aminouracil
6-amino-1-methyluracil
6-amino-1,3-dimethyluracil
4-chlorouracil
5-chlorouracil
5,6-dihydrouracil
5,6-dihydro-5-methyluracil
2-imidazolidone
1-methyl-2-imidazolidinone
1,3-dimethyl-2-imidazolidinone
4,5-dihydroxy-imidazolidin-2-one
1-(2-hydroxyethyl)-2-imidazolidinone
1-(2-hydroxypropyl)-2-imidazolidinone
1-(3-hydroxypropyl)-2-imidazolidinone
4,5-dihydroxy-1,3-dimethyl-imidazolidin-2-one
1,3-bis(2-hydroxyethyl)-2-imidazolidinone
2-imidazolidone-4-carboxylic acid
1-(2-aminoethyl)-2-imidazole
4-methyl-1,2,4-triazoline-3,5-dione
2,4-dihydroxy-6-methylpyrimidine
1-amino-4,5-dihydro-1H-tetrazol-5-one
hydantoin
1-methylhydantoin
5-methylhydantoin
5,5-dimethylhydantoin
5-ethylhydantoin
5-n-propylhydantoin
5-ethyl-5-methylhydantoin
5-hydroxy-5-methylhydantoin
5-hydroxymethylhydantoin
1-allylhydantoin
1-aminohydantoin
hydantoin-5-acetic acid
4-amino-1,2,4-triazolone-3,5-dione
hexahydro-1,2,4,5-tetrazine-3,6-dione
5-methyl-1,3,5-triazinon-2-one
1-methyltetrahydropyrimidin-2-one
2,4-dioxohexahydro-1,3,5-triazine
urazole
4-methylurazole
orotic acid
dihydroxyorotic acid
2,4,5-trihydroxypyrimidine
2-hydroxy-4-methylpyrimidine
4,5-diamino-2,6-dihydroxypyrimidine
barbituric acid
1,3-dimethylbarbituric acid
cyanuric acid
1-methyl-hexahydropyrimidine-2,4-dione
1,3-dimethyl-3,4,5,6-tetrahydro-2-1H-pyrimidinone
5-(hydroxymethyl-2,4-(1H,3H)-pyrimidinedione
2,4-dihydroxypyrimidine-5-carboxylic acid
6-azathymine
5-methyl-1,3,5-triazinan-2-one
N-carbamoylmaleamic acid and
alloxan monohydrate.

Preferably, the amine-generating compound is chosen from arginine, guanidine, urea and hydroxyethylurea, salts thereof, hydrates thereof or mixtures thereof.

Even more preferentially, the amine-generating compound is urea.

According to one embodiment, the composition of the invention comprises from 0.01% to 15% by weight of amine-generating derivatives corresponding to formula (II), salts thereof or hydrates thereof, preferably from 0.1% to 10% by weight and better still from 0.2% to 5% by weight relative to the total weight of the composition.

Preferably, the weight ratio of the dicarbonyl derivatives corresponding to formula (I) and/or one of the derivatives thereof and/or hydrate forms thereof and/or salts thereof/amine-generating derivatives corresponding to formula (II) or hydrate forms thereof and/or salts thereof may range from 0.1 to 100 and better still from 1 to 20.

According to a particular embodiment, the composition comprises at least one amphoteric or zwitterionic surfactant.

In particular, the amphoteric or zwitterionic surfactant(s), which are preferably non-silicone, which may be used in the present invention may especially be derivatives of optionally quaternized aliphatic secondary or tertiary amines, in which derivatives the aliphatic group is a linear or branched chain comprising from 8 to 22 carbon atoms, said amine derivatives containing at least one anionic group, for instance a carboxylate, sulfonate, sulfate, phosphate or phosphonate group.

Mention may be made in particular of (C₈-C₂₀)alkylbetaines, (C₈-C₂₀)alkylsulfobetaines, (C₈-C₂₀)alkylamido(C₃-C₈)alkylbetaines and (C₈-C₂₀)alkylamido(C₆-C₈)alkylsulfobetaines.

Among the optionally quaternized secondary or tertiary aliphatic amine derivatives that may be used, as defined above, mention may also be made of the compounds of respective structures (B1) and (B2) below:

Rₐ—C(O)—NH—CH₂—CH₂—N⁺(R_{b})(R_{c})—CH₂C(O)O⁻,M⁺,X⁻ (B1)
in which formula:
   Rₐ represents a C₁₀-C₃₀ alkyl or alkenyl group derived from an acid Rₐ—COOH preferably present in hydrolyzed copra oil, or a heptyl, nonyl or undecyl group;
   R_{b} represents a β-hydroxyethyl group; and
   R_{c} represents a carboxymethyl group;
   M⁺ represents a cationic counterion derived from an alkali metal or alkaline-earth metal, such as sodium, an ammonium ion or an ion derived from an organic amine, and
   X⁻ represents an organic or mineral anionic counterion, such as that chosen from halides, acetates, phosphates, nitrates, (C₁-C₄)alkyl sulfates, (C₁-C₄)alkyl- or (C₁-C₄)alkylaryl sulfonates, in particular methyl sulfate and ethyl sulfate; or alternatively M⁺ and X⁻ are absent;

Rₐ—C(O)—NH—CH₂—CH₂—N(B)(B′) (B2)
in which formula:
   B represents the group —CH₂—CH₂—O—X′;
   B′ represents the group —(CH₂)_{z}Y′, with z=1 or 2;
   X′ represents the group —CH₂—C(O)OH, —CH₂—C(O)OZ′, —CH₂—CH₂—C(O)OH or —CH₂—CH₂—C(O)OZ′, or a hydrogen atom;
   Y′ represents the group —C(O)OH, —C(O)OZ′, —CH₂—CH(OH)—SO₃H or the group —CH₂—CH(OH)—SO₃—Z′;
   Z′ represents a cationic counterion derived from an alkali metal or alkaline-earth metal, such as sodium, an ammonium ion or an ion derived from an organic amine;
   R_{a′} represents a C₁₀-C₃₀ alkyl or alkenyl group derived from an acid
   R_{a′}—C(O)OH, which is preferably present in copra oil or in hydrolyzed linseed oil, an alkyl group, especially a C₁₇ group and its iso form, or an unsaturated C₁₇ group.

The compounds of this type are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid and cocoamphodipropionic acid.

By way of example, mention may be made of the cocoamphodiacetate sold by the company Rhodia under the trade name Miranol® C2M Concentrate.

Use may also be made of compounds of formula (B′2):

Ra″-NH—CH(Y″)—(CH2)n-C(O)—NH—(CH2)n′-N(Rd)(Re) (B′2)
in which formula:
   Y″ represents the group —C(O)OH, —C(O)OZ″, —CH2-CH(OH)—SO3H or the group —CH2-CH(OH)—SO3-Z″;
   Rd and Re, independently of each other, represent a C1-C4 alkyl or hydroxyalkyl radical;
   Z″ represents a cationic counterion derived from an alkali metal or alkaline-earth metal, such as sodium, an ammonium ion or an ion derived from an organic amine;
   Ra″ represents a C10-C30 alkyl or alkenyl group derived from an acid
   Ra″-C(O)OH, which is preferably present in copra oil or in hydrolysed linseed oil;
n and n′ denote, independently of each other, an integer ranging from 1 to 3.
Among the compounds of formula (B′2), mention may be made of the compound classified in the CTFA dictionary under the name sodium diethylaminopropyl cocoaspartamide and sold by the company Chimex under the name Chimexane HB.

In accordance with a particular embodiment of the invention, the content of amphoteric or zwitterionic surfactant(s), when they are present, ranges from 0.05% to 30% by weight, preferably from 0.5% to 10% by weight and more preferably from 0.1% to 5% by weight, relative to the total weight of the composition.

The composition according to the invention may also comprise a cellulose-based polymer.

According to the invention, the term “cellulose-based” polymer means any polysaccharide compound having in its structure sequences of glucose residues linked together via β-1,4 bonds; in addition to unsubstituted celluloses, the cellulose derivatives may be anionic, cationic, amphoteric or nonionic. Thus, the cellulose-based polymers of the invention may be chosen from unsubstituted celluloses, including those in a microcrystalline form, and cellulose ethers. Among these cellulose-based polymers, cellulose ethers, cellulose esters and cellulose ester ethers are distinguished. Among the cellulose esters are mineral esters of cellulose (cellulose nitrates, sulfates, phosphates, etc.), organic cellulose esters (cellulose monoacetates, triacetates, amidopropionates, acetatebutyrates, acetatepropionates and acetatetrimellitates, etc.), and mixed organic/mineral esters of cellulose, such as cellulose acetatebutyrate sulfates and cellulose acetatepropionate sulfates. Among the cellulose ester ethers, mention may be made of hydroxypropylmethylcellulose phthalates and ethylcellulose sulfates.

The compositions according to the invention may be in any of the formulation forms conventionally used, and in particular in the form of an aqueous, alcoholic or aqueous-alcoholic, or oily solution or suspension; a solution or a dispersion of the lotion or serum type; an emulsion, in particular of liquid or semi-liquid consistency, of the O/W, W/O or multiple type; a suspension or emulsion of soft consistency of cream (O/W) or (W/O) type; an aqueous or anhydrous gel, or any other cosmetic form.

These compositions may be packaged in pump-action bottles or in aerosol containers, so as to apply the composition in vaporized (lacquer) form or in the form of a mousse. Such packaging forms are indicated, for example, when it is desired to obtain a spray or a mousse, for treating the hair. In these cases, the composition preferably comprises at least one propellant.

The compositions of the invention may be aqueous or anhydrous. They are preferably aqueous and then comprise water at a concentration ranging from 5% to 98%, better still from 5% to 90% and even better still from 10% to 90% by weight relative to the total weight of the composition.

The composition may in particular comprise one or more organic solvents, in particular water-soluble solvents, such as C₁-C₇ alcohols; mention may in particular be made of C₁-C₇ aliphatic monoalcohols, C₆-C₇ aromatic monoalcohols, C₃-C₇ polyols and C₃-C₇ polyol ethers, which may be employed alone or as a mixture with water.

The composition of the invention may also comprise at least one common cosmetic ingredient, chosen in particular from propellants; oils; solid fatty substances and in particular C₈-C₄₀ esters; C₈-C₄₀ acids; C₈-C₄₀ alcohols; surfactants other than those described previously; sunscreens; moisturizers; antidandruff agents; antioxidants; chelating agents; nacreous agents and opacifiers; plasticizers or coalescers; fillers; silicones and in particular polydimethylsiloxanes; polymeric or non-polymeric thickeners or gelling agents other than the cellulose-based polymers already mentioned; emulsifiers; polymers, in particular conditioning or styling polymers; fragrances; basifying agents such as sodium hydroxide or acidifying agents; silanes; crosslinking agents. The composition can, of course, comprise several cosmetic ingredients appearing in the above list.

Depending on their nature and the purpose of the composition, the normal cosmetic ingredients can be present in normal amounts which can be easily determined by those skilled in the art and which can be, for each ingredient, between 0.01% and 80% by weight. Those skilled in the art will take care to choose the ingredients included in the composition, and also the amounts thereof, such that they do not harm the properties of the compositions of the present invention.

The pH of the composition is preferably less than 4, and preferentially ranges from 1 to 3, better still from 1.5 to 3 and even better still from 1.7 to 3.

It may be adjusted to the desired value by means of acidifying and/or basifying agents usually used for treating keratin fibers.

The basifying agent may be chosen from mineral or organic or hybrid alkaline agents, or mixtures thereof.

The mineral alkaline agent(s) are preferably chosen from aqueous ammonia, alkali metal carbonates or bicarbonates such as sodium or potassium carbonate and sodium or potassium bicarbonate, sodium hydroxide or potassium hydroxide, or mixtures thereof.

The organic alkaline agent(s) are preferably chosen from organic amines with a pKb at 25° C. of less than 12, preferably less than 10 and even more advantageously less than 6. It should be noted that it is the pKb corresponding to the function of highest basicity.

Mention may be made, as hybrid compounds, of the salts of the abovementioned amines with acids, such as carbonic acid or hydrochloric acid.

The organic alkaline agent(s) are chosen, for example, from amine derivatives such as alkanolamines, oxyethylenated and/or oxypropylenated ethylenediamines, amino acids of amines such as 1,3-diaminopropane, 1,3-diamino-2-propanol, spermine or spermidine.

The term “alkanolamine” means an organic amine comprising a primary, secondary or tertiary amine function, and one or more linear or branched C₁-C₈ alkyl groups bearing one or more hydroxyl radicals.

Sodium hydroxide is in particular suitable for use in the invention.

The acidifying agent may be chosen from mineral or organic acids, for instance hydrochloric acid, phosphoric acid or lactic acid.

The composition according to the invention is preferably in the form of styling or care gels, care lotions or creams, conditioners, masks or sera.

The composition according to the invention may be obtained by mixing several compositions.

The process of the invention comprises the application of the composition described previously, followed by a step of straightening the hair with an iron, preferably at a temperature of at least 150° C. The straightening iron is known in the prior art. It consists in straightening the hair with flat heating tongs, which are generally metallic. The straightening irons are generally used at a temperature ranging from 150 to 250° C.

The process of the invention may comprise other intermediate steps aimed at improving the straightening of the hair.

According to a particular embodiment, the process of the invention comprises the application of the composition of the invention to dry hair, and a time of contact of the composition with the hair ranging from 10 to 60 minutes and preferably from 20 to 40 minutes. After this leave-on time, straightening with a brush and with a hairdryer (blow-drying) is performed. The hair is then straightened with a straightening iron at a temperature ranging from 150 to 250° C. and preferably from 210 to 230° C.

The process of the invention may comprise the application of other hair agents as a pretreatment or post-treatment. In particular, it may comprise the application of a conditioning care product as a post-treatment.

According to another embodiment, the hair straightening process comprises a step of washing the hair and then of drying with a hairdryer before applying the composition of the invention. According to this particular embodiment, the steps described above are then encountered, such as the contact time of the composition, the straightening with a straightening iron, the application of a conditioning agent and the rinsing, all these steps possibly being performed independently of each other, blow-drying possibly being intercalated between the contact of the composition according to the invention and the straightening with the iron. According to one particular embodiment, the straightening with the straightening iron is performed in several passes on the hair, in general 8 to 10 passes.

The process of the present invention is preferably performed without a step of permanent reshaping at basic pH or based on a reducing agent.

### EXAMPLES

The following compositions are prepared, compositions 1, 2 and 3 being compositions in accordance with the invention and compositions C1 to C6 being comparative examples.

### Exemplary Embodiments

| | 1 | C1 | C2 | C3 | 2 | 3 | C4 | C5 | C6 |
|---|---|---|---|---|---|---|---|---|---|
| Glyoxylic acid | 5 g | 5 g | — | — | 5 g | — | — | — | — |
| Pyruvic acid | — | — | — | — | — | 8 g | 8 g | — | — |
| Urea | 0.4 g | — | 0.4 g | 0.4 g | — | — | — | — | — |
| Hydroxyethylurea | — | — | — | — | — | 3 g | | 3 g | 3 g |
| Guanidine carbonate | — | — | — | — | 0.5 g | — | — | — | — |
| Sodium | qs | qs | — | qs | qs | qs | qs | — | qs |
| hydroxide/hydrochloric | pH | pH | | pH | pH | pH | pH | | pH |
| acid | 2.2 | 2.2 | | 2.2 | 2.2 | 2.2 | 2.2 | | 2.2 |
| Water | qs 100 g | qs 100 g | qs 100 g | qs 100 g | qs 100 g | qs 100 g | qs 100 g | qs 100 g | qs 100 g |

The compositions are applied to curly hair, which may be natural or dyed, or sensitized by a prior bleaching step, at a rate of 1 g per 2 g of hair. After 15 minutes, the hair is rinsed, dried with a hairdryer (blow-drying) and then straightened by treating with flat tongs heated to 210° C. It is subsequently shampooed to examine the persistence of the straightening effects and of modification of the mechanical and cosmetic properties of the fibers.

Compositions C2 and C3 do not make it possible to obtain permanent straightening. Compositions 1 and 2 according to the invention make it possible to obtain straightening properties superior to those obtained with composition C1, in terms of curl relaxation, protection of the natural or artificial color, resistance of the fibers to mechanical stresses (pulling, rubbing, twisting), sheen, smooth feel and smooth look.

Composition 3 according to the invention makes it possible to obtain straightening properties superior to those obtained with composition C4, C5 or C6.

1. A cosmetic composition comprising:
one or more dicarbonyl compounds corresponding to formula (I) below, and/or derivatives thereof and/or hydrates thereof and/or salts thereof: 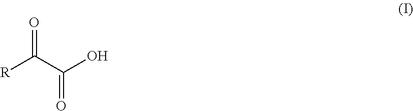
in which formula (I):
R represents an atom or group chosen from i) hydrogen, ii) carboxy —C(O)—OH, iii) linear or branched C₁-C₆ alkyl which is optionally substituted, preferably with at least one hydroxyl —OH radical, carboxy —C(O)—OH radical or halogen such as Br; iv) optionally substituted phenyl, v) optionally substituted benzyl, iv) and v) preferably being optionally substituted with at least one —OH or —C(O)—OH radical; vi) an indolyl radical and vii) an imidazolylmethyl radical and tautomers thereof such as 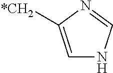
with * representing the part linked to the rest of the molecule,
one or more amine-generating derivatives chosen from the compounds of formula (II) below, salts thereof, tautomers thereof or hydrates thereof: 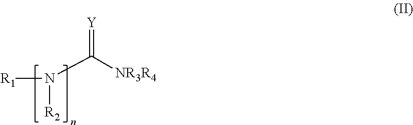
in which formula (II):
Y represents an atom or group chosen from i) oxygen, ii) sulfur, iii) N—R′; in particular, Y represents an oxygen atom or N—R′, more preferentially Y═O;
R′ representing an atom or group chosen from i) hydrogen, ii) a linear or branched, cyclic or acyclic C₁-C₅ alkyl or alkenyl radical, this radical being optionally substituted with one or more radicals chosen from the following radicals: hydroxyl, (di)(C₁-C₆)(alkyl)amino such as dimethylamino, carboxyl, halogen, C₆-C₁₈ aryl, carboxamide and N-methylcarboxamide; preferably, R′ represents a hydrogen atom or a group (C₁-C₆)alkyl optionally substituted with one or more (di)(C₁-C₆)(alkyl)amino and/or carboxyl radicals;
n=0 or 1, preferably 1,
R₁, R₂, R₃ and R₄, which may be identical or different, represent i) a hydrogen atom, a linear or branched, saturated or unsaturated, optionally substituted C₁-C₃₀ hydrocarbon-based chain, optionally interrupted and/or terminated at one or both of its ends with one or more divalent groups or combinations thereof chosen from:
—N(R)—, —N⁺(R)(R′)—, —O—, —S—, —C(O)—, —S(O)—, —S(O)₂— with R, R′, which may be identical or different, are chosen from hydrogen, C₁-C₄ alkyl, (C₁-C₄)hydroxyalkyl and (di)(C₁-C₄)(alkyl)aminoalkyl radicals; preferentially with one or more divalent groups or combinations thereof chosen from: —N(R)—, —O— and —C(O)—, —N(R)—, —N⁺(R)(R′)—, —O—, —S—, —C(O)—, —S(O)—, —S(O)₂—
an optionally substituted (hetero)cycloalkyl or optionally substituted (hetero)aryl radical;
or else
R₁ and R₂ and/or R₃ and R₄ form, together with the nitrogen atom that bears them, an optionally substituted heterocycloalkyl group or an optionally substituted heteroaryl group;
or else
R₁ and R₃ form, together with the nitrogen atom that bears them, an optionally substituted heterocycloalkyl group.

2. The composition as claimed in claim 1, in which R represents i) a hydrogen atom or ii) a linear or branched C₁-C₆ alkyl group optionally substituted with a carboxyl group.

3. The composition as claimed in either of the preceding claims, in which the dicarbonyl compound(s) corresponding to formula (I) and/or derivatives thereof and/or hydrates thereof and/or salts thereof are chosen from glyoxylic acid and pyruvic acid, a derivative thereof, salts thereof and hydrates thereof, preferably from glyoxylic acid, a derivative thereof and the hydrate forms of these compounds.

4. The composition as claimed in any one of the preceding claims, in which the dicarbonyl derivative(s) corresponding to formula (I) and/or derivatives thereof are chosen from glyoxylic acid esters, glyoxylic acid amides, glyoxylic acid (thio)acetals and hemi(thio)acetals, and glyoxylic acid ester (thio)acetals and hemi(thio)acetals.

5. The composition as claimed in claim 3, in which the glyoxylic acid is in its hydrate form.

6. The composition as claimed in any one of claims 1 to 5, comprising from 0.5% to 15% by weight of one or more dicarbonyl derivatives corresponding to formula (I) and/or derivatives thereof and/or hydrates thereof and/or salts thereof, preferably from 3% to 15% and preferentially from 5% to 10% by weight relative to the total weight of the composition.

7. The composition as claimed in any one of the preceding claims, in which the compound(s) of formula (II) are such that R₁, R₂, R₃ and R₄ represent, independently of each other:
(i) a hydrogen atom, (ii) a linear or branched, cyclic or acyclic C₁-C₅ alkyl or alkenyl radical, (iii) a C₁-C₅ alkoxy radical, (iv) a 5- to 18-membered (hetero)aryl radical, preferably C₆-C₁₈ aryl, (v) a 5- to 8-membered (hetero)cyclic radical; these radicals (ii) to (v) being optionally substituted in particular with one or more radicals chosen from the following radicals: hydroxyl, (C₁-C₄)alkyl, (di)(C₁-C₆)(alkyl)amino such as dimethylamino, carboxyl, halogen, C₆-C₁₈ aryl, carboxamide and N-methylcarboxamide;
it being understood that:
when R₁, R₂ and R₃ represent a hydrogen atom, R₄ may denote a radical from among: carboxamide, methoxy, ethoxy, 1,2,4-triazolyl, cyclopentyl, (C₁-C₆)alkylcarbonyl such as acetyl, (C₁-C₆)alkoxycarbonyl such as methoxycarbonyl or ethoxycarbonyl, —C(O)—C(H)═C(H)—C(O)—OH, phenyl optionally substituted with a chlorine atom or a hydroxyl, benzyl or 2,5-dioxo-4-imidazolidinyl radical;
when R₁ and R₃ represent a hydrogen atom, R₂ may represent a hydrogen atom or a methyl or ethyl radical and R₄ may represent an acetyl radical;
when R₁═R₂═H, R₃ and R₄ can form, with the nitrogen atom that bears them, a piperidine, 3-methylpyrazole, 3,5-dimethylpyrazole or maleimide ring;
R₁ and R₂ and also R₃ and R₄ can form, with the nitrogen atom that bears them, an imidazole ring;
R₁ and R₃ or R₂ and R₄ together possibly constituting a radical chosen from the following divalent radicals: —CH₂—CH₂—, —CH═CH—, —CH₂—C(O)—, —C(O)—N(H)—, —CH═N—, —C(O)—C(O)—, —CH(OH)—CH(OH)—, —[HO(O)C]CH—CH—, —CH(OH)—C(O)—, —CH₂—CH₂—CH₂—, —CH₂—N(H)—C(O)—, —CH═C(CH₃)—C(O)—, —N(H)—C(O)—N(H)—, —CH₂—CH₂—C(O)—, —CH₂—N(CH₃)—CH₂—, —N(H)—CH₂—N(H)—, —C(O)—CH(CH₃)—CH₂—, —C(O)—CH₂—C(O)—, —C(O)—N(H)—C(O)—, —C(O)—CH[C(O)OH)—CH₂—, —C(O)—CH═C[C(O)OH)]—, —C(O)—CH═C(CH₃)—, —C(O)—C(NH₂)═CH—, —C(O)—C(CH₃)═N—, —C(O)—CH═CH—, —C(O)—CH═N— and —C(O)—N═CH—;
as defined in claim 1, preferably Y═O.

8. The composition as claimed in any one of the preceding claims, in which the compound(s) of formula (II) are such that Y represents an atom or group chosen from oxygen and NH.

9. The composition as claimed in any one of the preceding claims, in which the compound(s) of formula (II) are such that R₁, R₂, R₃ and R₄, represent, independently of each other, an atom or group chosen from i) hydrogen, ii) C₁-C₃ alkyl, which is optionally substituted, preferably with a hydroxyl radical.

10. The composition as claimed in any one of the preceding claims, in which the compound(s) of formula (II) are such that R₁ and R₃ or R₂ and R₄ together constitute a saturated or unsaturated C₁-C₃ alkylene chain which may be optionally substituted with a hydroxyl group.

11. The composition as claimed in any one of the preceding claims, in which, when R′ represents a hydrogen atom, R₁ represents a hydrogen atom or a group —(CH₂)₃—CH(NH₂)—C(O)—OH.

12. The composition as claimed in any one of the preceding claims, in which the amine-generating compound(s) are chosen from urea or urea derivatives, salts thereof or hydrates thereof.

13. The composition as claimed in the preceding claim, in which urea or the urea derivatives are chosen from the compounds of formula (a) or (b), salts thereof, tautomers thereof or hydrates thereof: 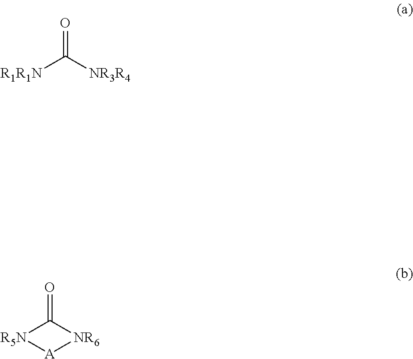
in which formulae (a) and (b):
R₁, R₂, R₃ and R₄ represent, independently of each other:
(i) a hydrogen atom or
(ii) a linear or branched, cyclic or acyclic C₁-C₅ alkyl or alkenyl radical, a C₁-C₅ alkoxy radical, (iii) a C₆-C₁₈ aryl radical, (iv) a 5- to 8-membered heterocyclic radical; these radicals being optionally substituted with one or more radicals chosen from the following radicals: hydroxyl, (C₁-C₄)alkyl, (di)(C₁-C₄)(alkyl)amino such as dimethylamino, carboxyl, halogen, C₆-C₁₈ aryl, carboxamide and N-methylcarboxamide;
it being understood that:
when R₁, R₂ and R₃ represent a hydrogen atom, R₄ may denote a radical from among: carboxamide, methoxy, ethoxy, 1,2,4-triazolyl, cyclopentyl, (C₁-C₆)alkylcarbonyl such as acetyl, (C₁-C₆)alkoxycarbonyl such as methoxycarbonyl or ethoxycarbonyl, —C(O)—C(H)═C(H)—C(O)—OH, phenyl optionally substituted with a chlorine atom or a hydroxyl, benzyl or 2,5-dioxo-4-imidazolidinyl radical;
when R₁ and R₃ represent a hydrogen atom, R₂ may represent a hydrogen atom or a methyl or ethyl radical and R₄ may represent an acetyl radical;
when R₁═R₂═H, R₃ and R₄ can form, with the nitrogen atom that bears them, a piperidine, 3-methylpyrazole, 3,5-dimethylpyrazole or maleimide ring;
R₁ and R₂ and also R₃ and R₄ can form, with the nitrogen atom that bears them, an imidazole ring;
R₅ and R₆ represent, independently of each other,
(iii) a hydrogen atom or
(iv) a linear or branched, cyclic or acyclic C₁-C₅ lower alkyl, acyl or alkenyl radical, a C₁-C₅ alkoxy radical, a C₆-C₁₈ aryl radical, a 5- to 8-membered heterocyclic radical; these radicals being optionally substituted with a radical chosen from the following radicals: hydroxyl, amino, dimethylamino, carboxyl, halogen, C₆-C₁₈ aryl, carboxamide and N-methylcarboxamide;
A is a divalent radical chosen from the following divalent radicals: —CH₂—CH₂—, —CH═CH—, —CH₂—C(O)—, —C(O)—N(H)—, —CH═N—, —C(O)—C(O)—, —CH(OH)—CH(OH)—, —[HO(O)C]CH—CH—, —CH(OH)—C(O)—, —CH₂—CH₂—CH₂—, —CH₂—N(H)—C(O)—, —CH═C(CH₃)—C(O)—, —N(H)—C(O)—N(H)—, —CH₂—CH₂—C(O)—, —CH₂—N(CH₃)—CH₂—, —N(H)—CH₂—N(H)—, —C(O)—CH(CH₃)—CH₂—, —C(O)—CH₂—C(O)—, —C(O)—N(H)—C(O)—, —C(O)—CH[C(O)OH)—CH₂—, —C(O)—CH═C[C(O)OH)]—, —C(O)—CH═C(CH₃)—, —C(O)—C(NH₂)═CH—, —C(O)—C(CH₃)═N—, —C(O)—CH═CH—, —C(O)—CH═N— and —C(O)—N═CH—.

14. The composition as claimed in the preceding claim, in which urea or the urea derivatives are chosen from the following compounds, salts thereof, tautomers thereof or hydrates thereof:
urea
methylurea
ethylurea
propylurea
n-butylurea
sec-butylurea
isobutylurea
tert-butylurea
cyclopentylurea
ethoxyurea
hydroxyethylurea
N-(2-hydroxypropyl)urea
N-(3-hydroxypropyl)urea
N-(2-dimethylaminopropyl)urea
N-(3-dimethylaminopropyl)urea
1-(3-hydroxyphenyl)urea
benzylurea
N-carbamoylmaleamide
N-carbamoylmaleamic acid
piperidinocarboxamide
1,2,4-triazol-4-ylurea
hydantoic acid
hydantoin
methyl allophanate
ethyl allophanate
acetylurea
hydroxyethyleneurea
2-(hydroxyethyl)ethyleneurea
diallylurea
chloroethylurea
N,N-dimethylurea
N,N-diethylurea
N,N-dipropylurea
cyclopentyl-1-methylurea
1,3-dimethylurea
1,3-diethylurea
1,3-bis(2-hydroxyethyl)urea
1,3-bis(2-hydroxypropyl)urea
1,3-bis(3-hydroxypropyl)urea
1,3-dipropylurea
ethyl-3-propylurea
sec-butyl-3-methylurea
isobutyl-3-methylurea
cyclopentyl-3-methylurea
N-acetyl-N′-methylurea
trimethylurea
butyl-3,3-dimethylurea
tetramethylurea
benzylurea
parabanic acid
1,2-dihydro-3-H-1,2,4-triazol-2-one
barbituric acid
uracil
1-methyluracil
3-methyluracil
5-methyluracil
1,3-dimethyluracil
5-azauracil
6-azauracil
5-fluorouracil
6-fluorouracil
1,3-dimethyl-5-fluorouracil
5-aminouracil
6-aminouracil
6-amino-1-methyluracil
6-amino-1,3-dimethyluracil
4-chlorouracil
5-chlorouracil
5,6-dihydrouracil
5,6-dihydro-5-methyluracil
2-imidazolidone
1-methyl-2-imidazolidinone
1,3-dimethyl-2-imidazolidinone
4,5-dihydroxy-imidazolidin-2-one
1-(2-hydroxyethyl)-2-imidazolidinone
1-(2-hydroxypropyl)-2-imidazolidinone
1-(3-hydroxypropyl)-2-imidazolidinone
4,5-dihydroxy-1,3-dimethyl-imidazolidin-2-one
1,3-bis(2-hydroxyethyl)-2-imidazolidinone
2-imidazolidone-4-carboxylic acid
1-(2-aminoethyl)-2-imidazole
4-methyl-1,2,4-triazoline-3,5-dione
2,4-dihydroxy-6-methylpyrimidine
1-amino-4,5-dihydro-1H-tetrazol-5-one
hydantoin
1-methylhydantoin
5-methylhydantoin
5,5-dimethylhydantoin
5-ethylhydantoin
5-n-propylhydantoin
5-ethyl-5-methylhydantoin
5-hydroxy-5-methylhydantoin
5-hydroxymethylhydantoin
1-allylhydantoin
1-aminohydantoin
hydantoin-5-acetic acid
4-amino-1,2,4-triazolone-3,5-dione
hexahydro-1,2,4,5-tetrazine-3,6-dione
5-methyl-1,3,5-triazinon-2-one
1-methyltetrahydropyrimidin-2-one
2,4-dioxohexahydro-1,3,5-triazine
urazole
4-methylurazole
orotic acid
dihydroxyorotic acid
2,4,5-trihydroxypyrimidine
2-hydroxy-4-methylpyrimidine
4,5-diamino-2,6-dihydroxypyrimidine
barbituric acid
1,3-dimethylbarbituric acid
cyanuric acid
1-methyl-hexahydropyrimidine-2,4-dione
1,3-dimethyl-3,4,5,6-tetrahydro-2-1H-pyrimidinone
5-(hydroxymethyl-2,4-(1H,3H)-pyrimidinedione
2,4-dihydroxypyrimidine-5-carboxylic acid
6-azathymine
5-methyl-1,3,5-triazinan-2-one
N-carbamoylmaleamic acid and
alloxan monohydrate.

15. The composition as claimed in any one of the preceding claims, in which the amine-generating derivative(s) corresponding to formula (II) are chosen from arginine, guanidine, urea and hydroxyethylurea, salts thereof and hydrates thereof, and more particularly urea, salts thereof and hydrates thereof.

16. The composition as claimed in one of the preceding claims, characterized in that the amine-generating derivative(s) corresponding to formula (II), salts thereof and hydrates thereof are present in an active material content ranging from 0.01% to 15% by weight, preferably from 0.1% to 10% by weight, preferably ranging from 0.2% to 5% by weight relative to the total weight of the composition.

17. The composition as claimed in any one of the preceding claims, in which the weight ratio of the dicarbonyl derivatives corresponding to formula (I) and/or one of the derivatives thereof and/or hydrate forms thereof and/or salts thereof/amine-generating derivatives corresponding to formula (II) or hydrate forms thereof and/or salts thereof ranges from 0.1 to 100 and better still from 1 to 20.

18. The composition as claimed in any one of the preceding claims, characterized in that it is aqueous and comprises water in a concentration preferably ranging from 5% to 98%, better still from 5% to 90% and even better still from 10% to 90% by weight relative to the total weight of the composition.

19. The composition as claimed in any one of the preceding claims, characterized in that it has a pH of less than 4, the pH preferentially ranging from 1 to 3, better still from 1.5 to 3 and even better still from 1.7 to 3.

20. The composition as claimed in any one of the preceding claims, characterized in that it results from the mixing of several compositions.

21. A process for straightening keratin fibers, especially the hair, which comprises the application to the hair of the composition as claimed in any one of the preceding claims, followed by a contact time ranging from 10 to 60 minutes and then a straightening step using a straightening iron at a temperature of at least 150° C., preferably ranging from 150 to 250° C.

22. The use of the composition as claimed in any one of claims 1 to 19, for straightening/relaxing keratin fibers, especially the hair.
